(19) United States
(12) Patent Application Publication
(10) Pub. No.: US 2006/0035389 A1
(43) Pub. Date: Feb. 16, 2006

(54) Immobilization of glycorproteins

(76) Inventors: Paul Peluso, Hayward, CA (US); Carolyn Bertozzi, Berkeley, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 10/514,577

(22) PCT Filed: May 15, 2003
(86) PCT No.: PCT/US03/15416

§ 371 Date: Jul. 11, 2005

Related U.S. Application Data
(60) Provisional application No. 60/380,923, filed on May 15, 2002.

(51) Int. Cl. G01N 33/53; C07K 16/46

(52) U.S. Cl. 436/547; 530/395; 530/391.1

(57) ABSTRACT

Methods and compositions for the immobilization of glycoproteins are presented herein. In addition, the present invention provides arrays of immobilized glycoproteins. The methods of immobilizing glycoproteins include oxidation of the glycosyl moiety.

### CROSS-REFERENCES TO RELATED APPLICATIONS

This application claims the benefit of U.S. Provisional Application No. 60/380,923 filed May 15, 2002 the disclosure of which is incorporated herein by reference in its entirety for all purposes.

### FIELD OF THE INVENTION

The invention relates to the field of biology, high throughput assays, proteomics, protein analysis, medical diagnostics, and the like.

### BACKGROUND OF THE INVENTION

Support-bound proteins are finding increasing utility, for example, in the search for small molecule modulators of the proteins in drug discovery programs. Recently, protein arrays have been described for high-throughput screening (see co-pending application Ser. Nos. 09/115,455, filed Jul. 14, 1998; 09/353,215, filed Jul. 14, 1999 and 09/353,555, filed Jul. 14, 1999; and related PCT published applications WO 00/04382, 00/04389 and 00/04390).

Applications Ser. Nos. 09/353,215 and 09/353,555 describe a number of hurdles that must be overcome to provide protein arrays of high quality which produce accurate and reproducible screening results. Typically, proteins must remain hydrated, be kept at ambient temperatures, and are very sensitive to the physical and chemical properties of the support materials. Thus, maintaining protein activity at the liquid-solid interface requires new immobilization strategies which address the sensitivity of the proteins to the environment and further can orient the protein in a manner which ensures accessibility of the protein active site to potentially interacting molecules.

The present invention addresses these and other considerations in the preparation and use of protein arrays.

### BRIEF SUMMARY OF THE INVENTION

The present invention provides methods and compositions for forming immobilized glycoproteins. Surprisingly, the present invention provides specifically oriented immobilized glycoproteins that exhibit high binding activity and are capable of forming a densely packed glycoprotein array.

In one aspect, the present invention provides a method for conjugating a glycoprotein to a compound. The method includes oxidizing the glycoprotein to form an oxidized glycoprotein. The oxidized glycoprotein is contacted with an aminooxy-functionalized compound. The oxidized glycoprotein is reacted with the aminooxy-functionalized compound to attach the oxidized glycoprotein to the aminooxy-functionalized compound.

In a second aspect, the present invention provides a method of forming an immobilized glycoprotein. The method includes contacting a glycosyl moiety covalently bound to the glycoprotein with an oxidizing agent to form an oxidized glycosyl moiety. The oxidized glycosyl moiety is contacted with an aminooxy-functionalized linking compound to form a glycoprotein-linking compound comprising an oxime bond. The glycoprotein-linking compound is contacted with an organic thinfilm to form an immobilized glycoprotein.

In a third aspect, the present invention provides a glycoprotein immobilized on an organic thinfilm. The glycoprotein contains a glycosyl moiety covalently bound to a linking compound through an oxime bond. The linking compound is bound to the organic thinfilm.

In a fourth aspect, the present invention provides an array of glycoproteins containing a plurality of glycoproteins arranged in discrete, known regions on portions of an organic thinfilm. Each of the glycoproteins contain a glycosyl moiety covalently bound to a linking compound through an oxime bond, wherein the linking compound is bound to the organic thinfilm.

### BRIEF DESCRIPTION OF THE DRAWINGS

FIG. 1 depicts an exemplary method of forming an organic thinfilm containing a biotin organic thinfilm functional group.

FIG. 2 depicts four immobilization strategies.

FIG. 3 depicts biotin dependent deposition of streptavidin and biotinylated-antibodies onto an alkanethiol biotin monolayer and the subsequent binding of a target analyte by the antibody.

FIG. 4 depicts the effects of the various linkage strategies on binding site densities and binding activities.

FIG. 5 depicts the effects of the various linkage strategies on MAB208 antibody forms and binding performance on the PLL-PEG Biotin Microarray Matrix.

FIG. 6 depicts the quantitation of the MAB208 antibody and sandwich assays.

FIG. 7 depicts a comparison of the four MAB208 antibody forms on the PLL PEG biotin microarray surface at 100 pm hIL8.

FIG. 8 Comparison of the four MAB602 antibody forms on the PLL PEG biotin microarray surface at 1 nM hIL2.

FIG. 9 depicts an exemplary chemical reaction for conjugating biotin to a glycoprotein.

### DETAILED DESCRIPTION OF THE INVENTION

Definitions

As used herein, the term “protein” means a polymer of amino acid residues linked together by peptide bonds. The term is meant to include proteins, polypeptides, and peptides of any size, structure, or function. Typically, however, a protein will be at least six amino acids long. In an exemplary embodiment, the protein is a short peptide that is at least about 10 amino acid residues long. A protein may be naturally occurring, recombinant, or synthetic, or any combination of these. A protein may also be just a fragment of a naturally occurring protein or peptide. A protein may be a single molecule or may be a multi-molecular complex. The term protein may also apply to amino acid polymers in which one or more amino acid residues is an artificial chemical analogue of a corresponding naturally occurring amino acid. An amino acid polymer in which one or more amino acid residues is an “unnatural” amino acid, not corresponding to any naturally occurring amino acid, is also encompassed by the use of the term “protein” herein.

The term “glycoprotein” refers to a protein covalently attached to a glycosyl moiety.

“Moiety” refers to a component, part or portion of a chemical molecule. A “glycosyl moiety” refers to an oligosaccharide that is typically covalently bonded to a protein to form a glycoprotein.

The term “antibody” means an immunoglobulin, whether natural or wholly or partially synthetically produced. All derivatives thereof which maintain specific binding ability are also included in the term. The term also covers any protein having a binding domain which is homologous or largely homologous to an immunoglobulin binding domain. These proteins may be derived from natural sources, or partly or wholly synthetically produced. An antibody may be monoclonal or polyclonal. In an exemplary embodiment, the antibody is a glycosylated antibody. The antibody may be a member of any immunoglobulin class, including any of the human classes: IgG, IgM, IgA, IgD, and IgE. In an exemplary embodiment, the antibody is of the IgG class.

The term “antibody fragment” refers to any derivative of an antibody which is less than full-length. In an exemplary embodiment, the antibody fragment retains at least a significant portion of the full-length antibody's specific binding ability. Examples of antibody fragments include, but are not limited to, Fab, Fab′, F(ab′)₂, scFv, Fv, dsFv diabody, and Fd fragments. The antibody fragment may be produced by any means. For instance, the antibody fragment may be enzymatically or chemically produced by fragmentation of an intact antibody or it may be recombinantly produced from a gene encoding the partial antibody sequence. Alternatively, the antibody fragment may be wholly or partially synthetically produced. The antibody fragment may optionally be a single chain antibody fragment. Alternatively, the fragment may comprise multiple chains which are linked together, for instance, by disulfide linkages. The fragment may also optionally be a multimolecular complex. A functional antibody fragment will typically comprise at least about 50 amino acids and more typically will comprise at least about 200 amino acids.

Single-chain Fvs (scFvs) are recombinant antibody fragments consisting of only the variable light chain (V_{L}) and variable heavy chain (V_{H}) covalently connected to one another by a polypeptide linker. Either V_{L} or V_{H} may be the NH₂-terminal domain. The polypeptide linker may be of variable length and composition so long as the two variable domains are bridged without serious steric interference. Typically, the linkers are comprised primarily of stretches of glycine and serine residues with some glutamic acid or lysine residues interspersed for solubility.

An “Fv” fragment is an antibody fragment which consists of one V_{H} and one V_{L} domain held together by noncovalent interactions. The term “dsFv” is used herein to refer to an Fv with an engineered intermolecular disulfide bond to stabilize the V_{H}-V_{L} pair.

A “F(ab′)₂” fragment is an antibody fragment essentially equivalent to that obtained from immunoglobulins (typically IgG) by digestion with an enzyme pepsin at pH 4.0-4.5. The fragment may be recombinantly produced.

A “Fab′” fragment is an antibody fragment essentially equivalent to that obtained by reduction of the disulfide bridge or bridges joining the two heavy chain pieces in the F(ab′)₂ fragment. The Fab′ fragment may be recombinantly produced.

A “Fab” fragment is an antibody fragment essentially equivalent to that obtained by digestion of immunoglobulins (typically IgG) with the enzyme papain. The Fab fragment may be recombinantly produced.

As used herein, the term “array” refers to an arrangement of entities in a pattern on a substrate. Although the pattern is typically a two-dimensional pattern, the pattern may also be a three-dimensional pattern. The term “substrate” refers to the bulk, underlying, and core material of the arrays of the invention.

The term “coating” means a layer that is either naturally or synthetically formed on or applied to the surface of the substrate. For instance, exposure of a substrate, such as silicon, to air results in oxidation of the exposed surface. In the case of a substrate made of silicon, a silicon oxide coating is formed on the surface upon exposure to air. In other instances, the coating is not derived from the substrate and may be placed upon the surface via mechanical, physical, electrical, or chemical means. An example of this type of coating would be a metal coating that is applied to a silicon or polymer substrate or a silicon nitride coating that is applied to a silicon substrate. Although a coating may be of any thickness, typically the coating has a thickness smaller than that of the substrate.

An “interlayer” is an additional coating or layer that is positioned between the coating and the substrate. Multiple interlayers may optionally be used together. The primary purpose of a typical interlayer is to aid adhesion between the first coating and the substrate. One such example is the use of a titanium or chromium interlayer to help adhere a gold coating to a silicon or glass surface. However, other possible functions of an interlayer are also anticipated. For instance, some interlayers may perform a role in the detection system of the array (such as a semiconductor or metal layer between a nonconductive substrate and a nonconductive coating).

An “organic thinfilm” is a thin layer of organic molecules which has been applied to a substrate or to a coating on a substrate if present. Typically, an organic thinfilm is less than about 20 nm thick. Optionally, an organic thinfilm may be less than about 10 nm thick. An organic thinfilm may be disordered or ordered. For instance, an organic thinfilm can be amorphous (such as a chemisorbed or spin-coated polymer) or highly organized (such as a Langmuir-Blodgett film or self-assembled monolayer). An organic thinfilm may be heterogeneous or homogeneous. In an exemplary embodiment, the organic thinfilm is a monolayer. In another exemplary embodiment, the organic thinfilm is a lipid bilayer. Optionally, the organic thinfilm may comprise a combination of more than one form of organic thinfilm. For instance, an organic thinfilm may comprise a lipid bilayer on top of a self-assembled monolayer. A hydrogel may also compose an organic thinfilm. The organic thinfilm will typically have functionalities exposed on its surface which serve to enhance the surface conditions of a substrate or the coating on a substrate in any of a number of ways. For instance, exposed functionalities of the organic thinfilm are typically useful in the binding or covalent immobilization of the proteins to the patches of the array. Alternatively, the organic thinfilm may bear functional groups (such as polyethylene glycol (PEG)) which reduce the non-specific binding of molecules to the surface. Other exposed functionalities serve to tether the thinfilm to the surface of the substrate or the coating. Particular functionalities of the organic thinfilm may also be designed to enable certain detection techniques to be used with the surface. Alternatively, the organic thinfilm may serve the purpose of preventing inactivation of a protein immobilized on a patch of the array or analytes which are proteins from occurring upon contact with the surface of a substrate or a coating on the surface of a substrate.

A “monolayer” is a single-molecule thick organic thinfilm or portion of an organic thinfilm. A monolayer may be disordered or ordered. A monolayer may optionally be a polymeric compound, such as a polynonionic polymer, a polyionic polymer, or a block-copolymer. For instance, the monolayer may be composed of a poly(amino acid) such as polylysine. In an exemplary embodiment, the monolayer is a self-assembled monolayer. One face of the self-assembled monolayer is typically composed of chemical functionalities on the termini of the organic molecules that are chemisorbed or physisorbed onto the surface of the substrate or, if present, the coating on the substrate. Examples of suitable functionalities of monolayers include biotin, the positively charged amino groups of poly-L-lysine for use on negatively charged surfaces, and thiols or disulfides for use on gold surfaces. Typically, the other face of the self-assembled monolayer is exposed and may bear any number of chemical functionalities (end groups). In an exemplary embodiment, the molecules of the self-assembled monolayer are highly ordered.

The term “alkyl,” by itself or as part of another substituent, means, unless otherwise stated, a straight or branched chain, or cyclic hydrocarbon radical, or combination thereof, which may be fully saturated, mono- or polyunsaturated and can include di- and multi-radicals, having the number of carbon atoms designated (i.e. C₁-C₁₀ means one to ten carbons). Examples of saturated hydrocarbon radicals include groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkylene” by itself or as part of another substituent means a divalent radical derived from an alkane, as exemplified by —CH₂CH₂CH₂CH₂—. Typically, an alkyl group will have from 1 to 24 carbon atoms. In an exemplary embodiment, the alkyl group has 10 to 24 carbon atoms. A “lower alkyl” or “lower alkylene” is a shorter chain alkyl or alkylene group, generally having eight or fewer carbon atoms.

The term “heteroalkyl,” by itself or in combination with another term, means, unless otherwise stated, a stable straight or branched chain, or cyclic hydrocarbon radical, or combinations thereof, consisting of the stated number of carbon atoms and from one to three heteroatoms selected from the group consisting of O, N, Si and S, and wherein the nitrogen and sulfur atoms may optionally be oxidized and the nitrogen heteroatom may optionally be quaternized. The heteroatom(s) O, N and S may be placed at any interior position of the heteroalkyl group. The heteroatom Si may be placed at any position of the heteroalkyl group, including the position at which the alkyl group is attached to the remainder of the molecule. Examples include —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. The term “heteroalkylene” by itself or as part of another substituent means a divalent radical derived from heteroalkyl, as exemplified by —CH₂—CH₂—S—CH₂CH₂— and —CH₂—S—CH₂—CH₂—NH—CH₂—. For heteroalkylene groups, heteroatoms can also occupy either or both of the chain termini. Still further, for alkylene and heteroalkylene linking groups, as well as all other linking groups described herein, no specific orientation of the linking group is implied.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. In addition, the term “cycloalkylene” and “heterocycloalkylene” by themselves or as part of another substituent means a divalent radical derived from a cycloalkyl or heterocycloalkyl, respectively.

The terms “halo” or “halogen,” by themselves or as part of another substituent, mean, unless otherwise stated, a fluorine, chlorine, bromine, or iodine atom.

The term “aryl,” employed alone or in combination with other terms (e.g., aryloxy, arylthioxy, arylalkyl) means, unless otherwise stated, an aromatic substituent which can be a single ring or multiple rings (up to three rings) which are fused together or linked covalently. The term “heteroaryl” refers to those aryl groups in which at least one of the rings contains from one to four heteroatoms selected from N, O, and S, wherein the nitrogen and sulfur atoms are optionally oxidized, and the nitrogen atom(s) are optionally quaternized. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-oxazolyl, 4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 2-pyridyl, 2-pyrimidyl, 5-benzothiazolyl, purinyl, 2-benzimidazolyl, 5-indolyl, 1-isoquinolyl, 2-quinoxalinyl, 3-quinolyl, and the like. Substituents for each of the above noted aryl ring systems are selected from the group of acceptable substituents described below. The term “arylene” by itself or as part of another substituent means a divalent radical derived from an aryl. In addition, the term “heteroarylene” by itself or as part of another substituent means a divalent radical derived from an aryl.

The terms “arylalkyl” and “arylheteroalkyl” are meant to include those radicals in which an aryl group is attached to an alkyl group (e.g., benzyl, phenethyl, pyridylmethyl and the like) or a heteroalkyl group (e.g., phenoxymethyl, 2-pyridyloxymethyl, 1-naphthyloxy-3-propyl, and the like). The arylalkyl and arylheteroalkyl groups will typically contain from 1 to 3 aryl moieties attached to the alkyl or heteroalkyl portion by a covalent bond or by fusing the ring to, for example, a cycloalkyl or heterocycloalkyl group. For arylheteroalkyl groups, a heteroatom can occupy the position at which the group is attached to the remainder of the molecule. For example, the term “arylheteroalkyl” is meant to include benzyloxy, 2-phenylethoxy, phenethylamine, and the like.

Each of the above terms (e.g., “alkyl,” “heteroalkyl” and “aryl”) are meant to include both substituted and unsubstituted forms of the indicated radical. Exemplary substituents for each type of radical are provided below.

Substituents for the alkyl and heteroalkyl radicals (including those groups often referred to as alkylene and heteroalkylene) can be a variety of groups selected from: —OR′, ═O, ═NR′, ═N—OR′, —NR′R″, —SR′, -halogen, —SiR′R″R′″, —OC(O)R′, —CO₂R′, —CONR′R″, —OC(O)NR′R″, —NR″C(O)R′, —NR″C(O)NR′R′″, —NR″C(O)₂R′, —NHC(NH₂)═NH, —NR′C(NH₂)═NH, —NH—C(NH₂)═NR′, —S(O)R′, —S(O)₂R′, —S(O)₂NR′R″, —CN and —NO₂ in a number ranging from zero to (2N+1), where N is the total number of carbon atoms in such radical. In an exemplary embodiment, substituted alkyl groups will have from one to six independently selected substituents, for example from one to four independently selected substituents. In the substituents listed above, R′, R″ and R′″ each independently refer to hydrogen, unsubstituted(C₁-C₈)alkyl and heteroalkyl, unsubstituted aryl, aryl substituted with 1-3 halogens, unsubstituted alkyl, alkoxy or thioalkoxy groups, or aryl-(C₁-C₄)alkyl groups. When R′ and R″ are attached to the same nitrogen atom, they can be combined with the nitrogen atom to form a 5-, 6-, or 7-membered ring. For example, —NR′R″ is meant to include 1-pyrrolidinyl and 4-morpholinyl.

Similarly, substituents for the aryl groups are varied and are selected from: -halogen, —OR′, —OC(O)R′, —NR′R″, —SR′, —R′, —CN, —NO₂, —CO₂R′, —CONR′R″, —OC(O)NR′R″, —NR″C(O)R′, —NR″C(O)₂R′, —NR″C(O)NR′R′″, —NH—C(NH₂)═NH, —NR′C(NH₂)═NH, —NH—C(NH₂)═NR′, —S(O)R′, —S(O)₂R′, —S(O)₂NR′R″, —N₃, —CH(Ph)₂, perfluoro(C₁-C₄)alkoxy, and perfluoro(C₁-C₄)alkyl, in a number ranging from zero to the total number of open valences on the aromatic ring system; and where R′ and R″ are independently selected from hydrogen, (C₁-C₈)alkyl and heteroalkyl, unsubstituted aryl, (unsubstituted aryl)-(C₁-C₄)alkyl, and (unsubstituted aryl)oxy-(C₁-C₄)alkyl. For example, substituted aryl groups will have from one to four independently selected substituents, or from one to three independently selected substituents, or from one to two independently selected substituents.

As used herein, the term “heteroatom” is meant to include oxygen (O), nitrogen (N), sulfur (S), boron (B) and silicon (Si).

The term “oxime bond” refers to a covalent linkage having the formula [symbol] —R′C═N—O— [symbol] , wherein R′ is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

The symbol [symbol] , whether utilized as a bond or displayed perpendicular to a bond indicates the point at which the displayed moiety is attached to the remainder of the molecule, solid support, etc.

Introduction

Protein arrays have the potential to revolutionize protein expression profiling. The amount of specific signal produced on a feature of such an array is related to the amount of analyte that is captured from a biological mixture by the immobilized glycoprotein. The amount of analyte captured is related to a variety of factors, including the density of proteins in the array and the fraction of the proteins able to bind the analytes. The present invention provides methods and compositions that enable proteins to be immobilized in a specific orientation allowing the immobilized glycoproteins to effectively bind analytes. In addition, the present invention provides arrays of specifically oriented immobilized glycoproteins wherein the specifically oriented immobilized proteins are densely packed.

In one embodiment, an array of specifically oriented immobilized antibodies are provided. The antibodies are immobilized by first oxidizing the vicinal diols of an antibody glycosyl moiety to form the respective aldehydes. The aldehydes of the glycosyl moiety are reacted with a biotin molecule functionalized with an aminooxy group. The reaction yields an antibody-biotin compound covalently bound through an oxime bond. Finally, the antibody-biotin compound is immobilized to an organic thinfilm comprising a streptavidin compound. The resulting array of specifically oriented immobilized antibodies is capable of binding approximately a 10-fold greater amount of target analytes than non-specifically oriented immobilized antibodies (see Examples below).

Methods of Immobilizing a Glycoprotein

In one aspect, the present invention provides a method for conjugating a glycoprotein to a compound. The method includes oxidizing the glycoprotein to form an oxidized glycoprotein. The oxidized glycoprotein is contacted with an aminooxy-functionalized compound. The oxidized glycoprotein is reacted with the aminooxy-functionalized compound to attach the oxidized glycoprotein to the aminooxy-functionalized compound.

In another aspect, the present invention provides a method of making an immobilized glycoprotein. The method involves oxidizing the glycosyl moiety of a glycoprotein with an oxidizing agent to form an oxidized glycosyl moiety. The oxidized glycosyl moiety is contacted with a linking compound containing a reactive aminooxy group. The oxidized glycosyl moiety reacts with the aminooxy group to form a glycoprotein-linking compound. The glycoprotein-linking compound is contacted with an organic thinfilm to form an immobilized glycoprotein.

Oxidizing the Glycosyl Moiety of a Glycoprotein

In one embodiment, the present invention provides methods of oxidizing a glycoprotein. Typically, an oxidizing agent is used to oxidize the glycosyl moiety of a glycoprotein. Oxidizing agents useful in the current invention include those capable of oxidizing a vicinal diol of a glycosyl moiety to an aldehyde. Useful oxidizing agents include, for example, metal oxyacids (e.g., chromates, permangates, osmonium tetraoxides, sodium periodates such as sodium meta peroidate), nitric and nitrous acids, halogens, ozone, di-oxygen, peroxides, peroxyacids, mild oxidizing reagents (e.g. silver oxide), and the like. In an exemplary embodiment, the oxidizing agent is sodium meta periodate.

A variety of glycosyl moieties are useful in the current invention. Glycosyl moieties are portions of intact oligosaccharides or intact oligosaccharides that are covalently linked to a protein. A variety of covalent linkages between the glycosyl moiety and the protein are useful in the current invention. In an exemplary embodiment, the glycosyl moiety is covalently linked through an asparagine side chain of the protein to from an N-linked glycosyl moiety. In another exemplary embodiment, the glycosyl moiety is covalently linked through a serine or threonine side chain of the protein to from an O-linked glycosyl moiety.

A variety of glycoproteins are useful in the present invention. Typically, glycoproteins are capable of specifically binding a target analyte in a mixture containing multiple analytes. Useful glycoproteins include those belonging to a receptor family (e.g. growth factor receptors, catecholamine receptors, amino acid derivative receptors, cytokine receptors, lectins), ligand family (e.g. cytokines, serpins), an enzyme family (e.g. proteases, kinases, phosphatases, ras-like GTPases, hydrolases), and transcription factors (e.g. steroid hormone receptors, heat-shock transcription factors, zinc-finger proteins, leucine-zipper proteins, homeodomain proteins). In an exemplary embodiment, the immobilized glycoproteins is an HIV proteases, a hepatitis C virus (HCV) proteases, a hormone receptor, neurotransmitter receptor, extracellular matrix receptor, an antibody, a DNA-binding protein, an intracellular signal transduction modulator or effectors, an apoptosis-related factor, a DNA synthesis factor, a DNA repair factor, a DNA recombination factor, or a cell-surface antigen. In an exemplary embodiment, the glycoprotein is a member of the IgG class of antibodies wherein the glycosyl moiety is covalently bonded to an asparagine residue.

A variety of target analytes are useful in the present invention, including, for example, nucleic acids, proteins, oligosaccharides, lipids, toxins, bacteria, and viruses. Target analytes are analytes to which the glycoprotein is capable of specifically binding. Typically, specific binding refers to binding interactions wherein the dissociation constant (K_{d}) is at least in the millimolar range. In an exemplary embodiment, the glycoprotein binds to the target analyte wherein the target analyte is in a mixture containing a plurality of non-target analytes.

The glycoprotein may be produced using a variety of methods. In an exemplary embodiment, the glycoprotein is produced recombinantly (see generally, Sambrook et al. MOLECULAR CLONING: A LABORATORY MANUAL, 2d ed. (1989) Cold Spring Harbor Laboratory Press, Cold Spring Harbor, N.Y., which is incorporated herein by reference) wherein the protein is glycosylated intracellularly using one or more glycosyltransferase enzymes. In another exemplary embodiment, the protein portion of the glycoprotein is produced using peptide synthesis techniques wherein the synthetic protein is glycosylated in vitro using one or more glycosyltransferase enzymes. In another embodiment, the protein portion of the glycoprotein is produced using peptide synthesis techniques wherein the synthetic protein is glycosylated by incorporating glycosylated amino acids into the protein during peptide synthesis.

Forming a Covalent Bond Between a Glycoprotein and a Linking Protein

In another embodiment, the present invention provides methods of forming a covalent bond between a glycoprotein and a linking compound yielding a glycoprotein-linking compound. The glycoprotein contains a glycosyl moiety oxidized by the methods disclosed above to produce a glycosyl moiety comprising an aldehyde group. The linking compound is functionalized with a reactive group capable of reacting with an aldehyde to produce a covalent bond.

A variety of reactive groups capable of reacting with an aldehyde to produce a covalent bond are useful in the current invention. Useful reactive groups include, for example, alkoxides (yielding the product of aldol addition), Grignard-type reactive groups, alcohol groups (yielding the corresponding acetal or hemiacetal), cyanide reactive groups, primary or secondary amines, hydrazines, phosphorus ylides, aminooxy reactive groups, and the like.

In an exemplary embodiment, the linking compound is functionalized with an aminooxy reactive group. The aminooxy reactive group has the formula L—R¹—O—NH₂, wherein L is a linking compound or portion thereof and R¹ is a substituted or unsubstituted alkylene, a substituted or unsubstituted heteroalkylene, a substituted or unsubstituted cycloalkylene, a substituted or unsubstituted heterocycloalkylene, a substituted or unsubstituted arylene, or a substituted or unsubstituted heteroarylene. In another exemplary embodiment, R¹ is a substituted or unsubstituted alkylene or a substituted or unsubstituted heteroalkylene. In another exemplary embodiment, R¹ is a substituted or unsubstituted 1 to 40 membered alkylene or a substituted or unsubstituted 1 to 40 membered heteroalkylene. In another exemplary embodiment, R¹ is a substituted or unsubstituted 1 to 20 membered alkylene or a substituted or unsubstituted 1 to 20 membered heteroalkylene. In another exemplary embodiment, R¹ is a substituted or unsubstituted 1 to 40 membered heteroalkylene. In another exemplary embodiment, R¹ is a substituted or unsubstituted 1 to 20 membered heteroalkylene.

Immobilization of a Glycoprotein on an Organic Thinfilm

In another embodiment, the present invention provides methods of immobilizing a glycoprotein on an organic thinfilm. The glycoprotein is covalently bound to a linking compound as described above to form a glycoprotein-linking compound. The linking compound portion of the glycoprotein-linking compound interacts with the organic thinfilm yielding an immobilized glycoprotein. Thus, the immobilized glycoprotein is attached to the organic thinfilm through the linking compound.

In an alternative exemplary embodiment, the linking compound is attached to an organic thinfilm prior to contacting the oxidized glycoprotein.

Organic thinfilms are defined above and described in detail in U.S. application Ser. No. 09/820,210, which is assigned to the same assignee as the present application and which is herein incorporated by reference in its entirety for all purposes. Organic thinfilms of the current invention are supported by a substrate and, optionally, a coating between the substrate and the organic thinfilm and, optionally, an interlayer between the coating and the substrate.

The substrate may be either organic or inorganic, biological or non-biological, or any combination of these materials. The substrate of the invention can comprise a material selected from a group consisting of silicon, silica, quartz, glass (e.g. silicon oxide), controlled pore glass, carbon, alumina, titania, tantalum oxide, germanium, silicon nitride, zeolites, and gallium arsenide. Many metals such as gold, platinum, aluminum, copper, titanium, and their alloys are also options for substrates. In addition, many ceramics and polymers may also be used as substrates. Polymers which may be used as substrates include, but are not limited to, the following: polystyrene; poly(tetra)fluoroethylene (PTFE); polyvinylidenedifluoride; polycarbonate; polymethylmethacrylate; polyvinylethylene; polyethyleneimine; poly(etherether)ketone; polyoxymethylene (POM); polyvinylphenol; polylactides; polymethacrylimide (PMI); polyatkenesulfone (PAS); polypropylene; polyethylene; polyhydroxyethylmethacrylate (HEMA); polydimethylsiloxane; polyacrylamide; polyimide; and block-copolymers.

The coating is optionally a metal film. Possible metal films include aluminum, chromium, titanium, tantalum, nickel, stainless steel, zinc, lead, iron, copper, magnesium, manganese, cadmium, tungsten, cobalt, and alloys or oxides thereof. In an exemplary embodiment, the metal film is a noble metal film. Noble metals that may be used for a coating include, but are not limited to, gold, platinum, silver, and copper. In another exemplary embodiment, the coating comprises gold or a gold alloy. Electron-beam evaporation may be used to provide a thin coating of gold on the surface of the substrate. In another exemplary embodiment, the metal film is from about 50 nm to about 500 nm in thickness. In an alternative embodiment, the metal film is from about 1 nm to about 1 μm in thickness. In alternative embodiments, the coating comprises a composition selected from the group consisting of silicon, silicon oxide, titania, tantalum oxide, silicon nitride, silicon hydride, indium tin oxide, magnesium oxide, alumina, glass, hydroxylated surfaces, and polymers.

A variety of different organic thinfilms are suitable for use in the present invention. Methods for the formation of organic thinfilms include in situ growth from the surface, deposition by physisorption, spin-coating, chemisorption, self-assembly, or plasma-initiated polymerization from gas phase. For instance, a hydrogel composed of a material such as dextran can serve as a suitable organic thinfilm on the patches of the array. In an exemplary embodiment of the invention, the organic thinfilm is a lipid bilayer. In another exemplary embodiment, the organic thinfilm of each of the patches of the array is a monolayer. A monolayer of polyarginine or polylysine adsorbed on a negatively charged substrate or coating is one option for the organic thinfilm. Another option is a disordered monolayer of tethered polymer chains. In another exemplary embodiment, the organic thinfilm contains self-assembled monolayer. A monolayer of polylysine is one option for the organic thinfilm. See Wagner, et al. U.S. patent application Ser. Nos. 09/353,215 and 09/353,555, both of which are herein incorporated by reference in their entirety for all purposes including methods and devices for displaying compounds in an array. In exemplary embodiments, the coating, or the substrate itself if no coating is present, should be compatible with the chemical or physical adsorption of the organic thinfilm on its surface.

An organic thinfilm of the present invention typically contains an organic thinfilm functional group capable of interacting with the linking compound portion of a glycoprotein-linking compound. Thus, the organic thinfilm functional group is chosen to correspond with the linking compound portion to provide a organic thinfilm functional group/linking compound binding pair.

A variety of organic thinfilm functional groups are useful for forming a non-covalent, covalent or reversibly covalent association between the organic thinfilm and the linking compound portion of the glycoprotein-linking compound. Examples of suitable organic thinfilm functional group/linking compound portion pairs are set forth in Table 1 below.

TABLE 1

| Organic Thinfilm | Linking Compound Portion of the |
|---|---|
| Functional Group | Glycoprotein-Linking Compound |
| His (6-8 aa) | NTA (Nitrilotriacetic acid, with |
| | a metal such as Ni, Co, Fe, Cu) |
| GST (220 aa) | GSH (Glutathione, 3 amino acids) |
| S (104 aa) | S-peptide (15 amino acids) |
| PKA peptide (5 amino acids) | PKA |
| HA peptide (9 amino acids) | HA |
| Arg (6-10 Arg) | OligoGlutamic acid (10-15 amino acids) |
| MBP (360 aa) | Maltose |
| GBD | Galactose |
| CBD (107-156 aa) | Cellulose |
| Streptavidin | Biotin |
| Thioredoxin | OligoGlutamic acid (10-15 amino acids) |
| Asp (6-10 Asp) | OligoArginine (10-15 amino acids) |
| KSI (125 aa) | OligoPhenylalanine, or OligoLeucine |
| | (10-30 amino acids) |

In an exemplary embodiment, the organic thinfilm functional group contains a heterofunctional group of the type described in U.S. application Ser. No. 09/820,210, which is assigned to the same assignee as the present application and which is herein incorporated by reference in its entirety for all purposes.

The non-covalent, covalent or reversibly covalent bond between the linking compound portion of the glycoprotein-linking compound and the organic thinfilm is typically stable to conditions in which the glycoprotein is capable of binding to a target analyte. Therefore, a variety of bonding interactions are useful in the current invention, including covalent interactions, ionic interactions, hydrogen bonding interactions, Van der Waals interactions, dipole-dipole interactions and the like.

In an exemplary embodiment, the organic thinfilm is a monolayer containing a biotin covalently bound to an unsymmetrical alkane disulfide (see Examples below). The disulfide portion is complexed to a gold coating wherein the gold coating is supported by a glass (silicon oxide) substrate. The organic thinfilm further comprises a streptavidin organic thinfilm functional group bound to the biotin portion of the organic thinfilm. The streptavidin organic thinfilm functional group is capable of binding-containing linking compound portion of the glycoprotein-linking compound thereby forming an immobilized glycoprotein.

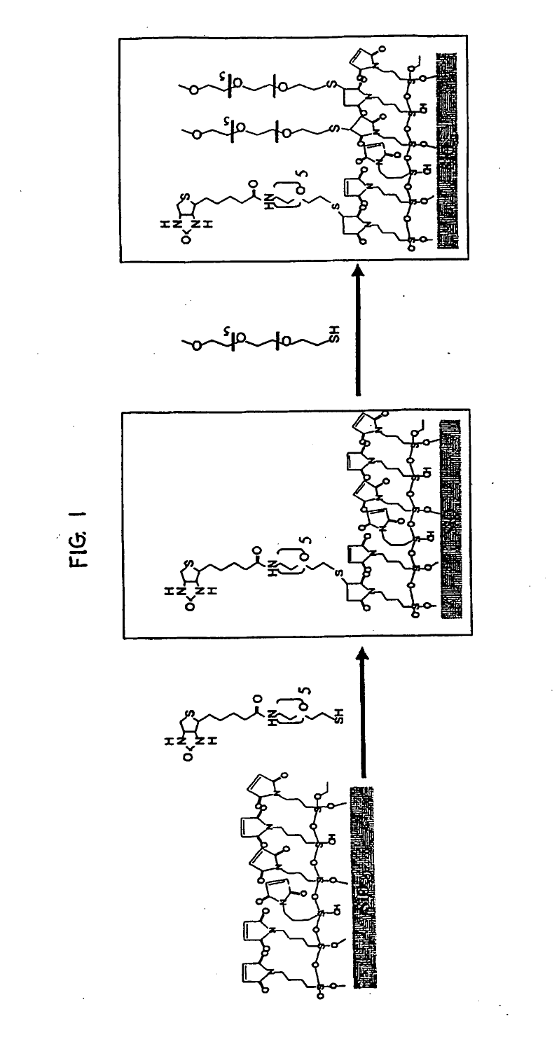

In another exemplary embodiment, the organic thinfilm is a monolayer containing a biotin covalently bound to N-alkylene succinimide through a thioether bond formed through a biotin-sulfhydryl maleimide reaction (FIG. 1). The N-alkylene succinimide is covalently bound to a silicon oxide substrate. The organic thinfilm further comprises a streptavidin organic thinfilm functional group bound to the biotin portion of the organic thinfilm. The streptavidin organic thinfilm functional group is capable of binding a biotin-containing linking compound portion of the glycoprotein-linking compound thereby forming an immobilized glycoprotein.

In another exemplary embodiment, the organic thinfilm is covalently bound to a substrate using the Staudinger ligation method, which is discussed in detail in U.S. Application Publication No. 2002/0016003, which is incorporated by reference herein in its entirety. Typically, the substrate contains an azide functionality that reacts with a methyl ester functionality of a precursor organic thinfilm yielding a substrate coupled to an organic thinfilm via an amide linkage. The amide linked organic thinfilm may comprise a variety of organic thinfilm functional groups as describes above.

Immobilized Glycoproteins

In a third aspect, the present invention provides a glycoprotein immobilized on an organic thinfilm. The glycoprotein contains a glycosyl moiety covalently bound to a linking compound through an oxime bond (defined above). The linking compound is bound to the organic thinfilm.

The immobilized glycoprotein compositions are formed using the methodologies of the present invention. Thus, the oxime bonds of the immobilized glycoprotein compositions are formed between the aldehyde functionality of a glycosyl moiety (covalently attached to a glycol protein) and a linking compound as described herein.

Oxime bonds of the current invention have the formula [symbol] —R²C═N—O— [symbol] , wherein R² is a hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In an exemplary embodiment, R² is a substituted or unsubstituted alkyl or a substituted or unsubstituted heteroalkyl. In another exemplary embodiment, the oxime bond is a substituted or unsubstituted heteroalkyl. The symbol [symbol] indicates the point at which the oxime bond is attached to a glycosyl moiety and a linking compound. Typically, the glycoprotein is covalently linked through the carbon of the oxime bond and the linking compound is covalently linked through the oxygen of the oxime bond.

Glycoprotein Arrays

In a fourth aspect, the present invention provides an array of glycoproteins containing a plurality of glycoproteins arranged in discrete, known regions on portions of an organic thinfilm. Each of the glycoproteins contain a glycosyl moiety covalently bound to a linking compound through an oxime bond, wherein the linking compound is bound to the organic thinfilm. Useful arrays and methods of making the arrays are discussed in detail in U.S. application Ser. No. 09/820,210, which is herein incorporated by reference in its entirety for all purposes.

In one embodiment, the present invention provides an array of glycoproteins containing a substrate, at least one organic thinfilm on some or all of the substrate surface, and a plurality of patches arranged in discrete, known regions on portions of the substrate surface covered by organic thinfilm, wherein each of said patches comprises a protein immobilized on the underlying organic thinfilm. The array optionally contains an interlayer between the substrate and coating.

In most cases, the array will comprise at least about ten patches. In an exemplary embodiment, the array comprises at least about 50 patches. In another exemplary embodiment the array comprises at least about 100 patches. In alternative exemplary embodiments, the array of proteins may comprise more than 10³, 10⁴ or 10⁵ patches.

In an exemplary embodiment, the area of surface of the substrate covered by each of the patches is no more than about 0.25 mm². In another exemplary embodiment, the area of the substrate surface covered by each of the patches is between about 1 μm² and about 10,000 μm². In another exemplary embodiment, each patch covers an area of the substrate surface from about 100 μm² to about 2,500 μm². In an alternative embodiment, a patch on the array may cover an area of the substrate surface as small as about 2,500 nm², although patches of such small size are generally not necessary for the use of the array.

The patches of the array may be of any geometric shape. For instance, the patches may be rectangular or circular. The patches of the array may also be irregularly shaped.

The distance separating the patches of the array can vary. For example, the patches of the array are separated from neighboring patches by about 1 μm to about 500 μm. Typically, the distance separating the patches is roughly proportional to the diameter or side length of the patches on the array if the patches have dimensions greater than about 10 μm. If the patch size is smaller, then the distance separating the patches will typically be larger than the dimensions of the patch.

In an exemplary embodiment of the array, the patches of the array are all contained within an area of about 1 cm² or less on the surface of the substrate. In one exemplary embodiment of the array, therefore, the array comprises 100 or more patches within a total area of about 1 cm² or less on the surface of the substrate. Alternatively, an exemplary array comprises 10³ or more patches within a total area of about 1 cm² or less. An exemplary array may even optionally comprise 10⁴ or 10⁵ or more patches within an area of about 1 cm² or less on the surface of the substrate. In other embodiments of the invention, all of the patches of the array are contained within an area of about 1 m² or less on the surface of the substrate.

Typically, only one type of glycoprotein is immobilized on each patch of the array. In an exemplary embodiment of the array, the glycoprotein immobilized on one patch differs from the glycoprotein immobilized on a second patch of the same array. In such an embodiment, a plurality of different glycoproteins are present on separate patches of the array. Typically the array comprises at least about ten different glycoproteins. In an exemplary embodiment, the array comprises at least about 50 different glycoproteins. In another exemplary embodiment, the array comprises at least about 100 different glycoproteins. Alternative exemplary arrays comprise more than about 10³ different glycoproteins or more than about 10⁴ different glycoproteins. The array may even optionally comprise more than about 10⁵ different glycoproteins.

In one embodiment of the array, each of the patches of the array contains a different glycoprotein. For instance, an array comprising about 100 patches could comprise about 100 different glycoproteins. Likewise, an array of about 10,000 patches could comprise about 10,000 different glycoproteins. In an alternative embodiment, however, each different glycoprotein is immobilized on more than one separate patch on the array. For instance, each different glycoprotein may optionally be present on two to six different patches. An array of the invention, therefore, may comprise about three-thousand glycoprotein patches, but only comprise about one thousand different glycoproteins since each different protein is present on three different patches.

In another embodiment of the present invention, although the glycoprotein of one patch is different from that of another, the glycoproteins are related. In an exemplary embodiment, the two different glycoproteins are members of the same protein family. The different glycoproteins on the invention array may be either functionally related or just suspected of being functionally related. In another embodiment of the invention array, however, the function of the immobilized glycoproteins may be unknown. In this case, the different glycoproteins on the different patches of the array share a similarity in structure or sequence or are simply suspected of sharing a similarity in structure or sequence. Alternatively, the immobilized glycoproteins may be just fragments of different members of a protein family.

In another exemplary embodiment, the immobilized glycoproteins are all HIV proteases or hepatitis C virus (HCV) proteases. In other exemplary embodiments of the invention, the immobilized proteins on the patches of the array are all hormone receptors, neurotransmitter receptors, extracellular matrix receptors, antibodies, DNA-binding proteins, intracellular signal transduction modulators and effectors, apoptosis-related factors, DNA synthesis factors, DNA repair factors, DNA recombination factors, or cell-surface antigens.

In an exemplary embodiment, the glycoprotein immobilized on each patch is an antibody or antibody fragment. The antibodies or antibody fragments of the array may optionally be single-chain Fvs, Fab fragments, Fab′ fragments, F(ab′)2 fragments, Fv fragments, dsFvs diabodies, Fd fragments, full-length, antigen-specific polyclonal antibodies, or full-length monoclonal antibodies. In another exemplary embodiment, the immobilized glycoproteins on the patches of the array are monoclonal antibodies, Fab fragments or single-chain Fvs.

In another exemplary embodiment of the invention, the glycoproteins immobilized to each patch of the array are protein-protein tag combinations.

In an alternative embodiment of the invention array, the glycoproteins on different patches are identical.

Biosensors, micromachined devices, and diagnostic devices that comprise the protein arrays of the invention are also contemplated by the present invention.

The physical structure of the glycoprotein arrays will typically comprise a substrate and, optionally, a coating or organic thinfilm or both.

The substrate on which the patches reside may also be a combination of any of the aforementioned substrate materials. Exemplary substrates for the array include silicon, silica, glass, and polymers. In an exemplary embodiment, the substrate is transparent or translucent. In another exemplary embodiment, the portion of the surface of the substrate on which the patches reside is flat and firm or semi-firm. However, the array of the present invention need not necessarily be flat or entirely two-dimensional. Significant topological features may be present on the surface of the substrate surrounding the patches, between the patches or beneath the patches. For instance, walls or other barriers may separate the patches of the array. In another exemplary embodiment of the invention array, the surface of the coating is atomically flat. In this embodiment, the mean roughness of the surface of the coating is less than about 5 angstroms for areas of at least 25 μm². In another exemplary embodiment, the mean roughness of the surface of the coating is less than about 3 angstroms for areas of at least 25 μm². The ultraflat coating can optionally be a template-stripped surface as described in Heguer et al., Surface Science, 1993, 291:39-46 and Wagner et al., Langmuir, 1995, 11:3867-3875, both of which are incorporated herein by reference.

It is contemplated that the coatings of many arrays will require the addition of at least one adhesion layer between said coating and the substrate. Typically, the adhesion layer will be at least 6 angstroms thick and may be much thicker. For instance, a layer of titanium or chromium may be desirable between a silicon wafer and a gold coating. In an alternative embodiment, an epoxy glue such as Epo-tek 377®, Epo-tek 301-2®, (Epoxy Technology Inc., Billerica, Mass.) may aid adherence of the coating to the substrate. Determinations as to what material should be used for the adhesion layer would be obvious to one skilled in the art once materials are chosen for both the substrate and coating. In other embodiments, additional adhesion mediators or interlayers may be necessary to improve the optical properties of the array, for instance, in waveguides for detection purposes.

Deposition or formation of the coating (if present) on the substrate is performed prior to the formation of the organic thinfilm thereon. Several different types of coating may be combined on the surface. The coating may cover the whole surface of the substrate or only parts of it. The pattern of the coating may or may not be identical to the pattern of organic thinfilms used to immobilize the proteins. In one embodiment of the invention, the coating covers the substrate surface only at the site of the patches of the immobilized protein(s). Techniques useful for the formation of coated patches on the surface of the substrate which are organic thinfilm-compatible are well known to those of ordinary skill in the art. For instance, the patches of coatings on the substrate may optionally be fabricated by photolithography, micromolding (PCT Publication WO 96/29629), wet chemical or dry etching, or any combination of these.

The organic thinfilm on which each of the patches of proteins is immobilized forms a layer either on the substrate itself or on a coating covering the substrate. In an exemplary embodiment, the organic thinfilm on which the proteins of the patches are immobilized is less than about 20 nm thick. In some embodiments of the invention, the organic thinfilm of each of the patches may be less than about 10 nm thick.

A variety of techniques may be used to generate patches of organic thinfilm on the surface of the substrate or on the surface of a coating on the substrate. These techniques are well known to those skilled in the art and will vary depending upon the nature of the organic thinfilm, the substrate, and the coating if present. The techniques will also vary depending on the structure of the underlying substrate and the pattern of any coating present on the substrate. For instance, patches of a coating which is highly reactive with an organic thinfilm may have already been produced on the substrate surface. Arrays of patches of organic thinfilm can optionally be created by microfluidics printing, microstamping (U.S. Pat. Nos. 5,512,131 and 5,731,152), or microcontact printing (PCT Publication WO 96/29629). Subsequent immobilization of glycoproteins to the monolayer patches results in two-dimensional arrays of the agents. Inkjet printer heads provide another option for patterning monolayer molecules, or components thereof, or other organic thinfilm components to nanometer or micrometer scale sites on the surface of the substrate or coating (Lemmo et al., Anal Chem., 1997, 69:543-551; U.S. Pat. Nos. 5,843,767 and 5,837,860). In some cases, commercially available arrayers based on capillary dispensing (for instance, OmniGrid™ from Genemachines, Inc, San Carlos, Calif., and High-Throughput Microarrayer from Intelligent Bio-Instruments, Cambridge, Mass.) may also be of use in directing components of organic thinfilms to spatially distinct regions of the array.

Diffusion boundaries between the patches of proteins immobilized on organic thinfilms such as self-assembled monolayers may be integrated as topographic patterns (physical barriers) or surface functionalities with orthogonal wetting behavior (chemical barriers). For instance, walls of substrate material or photoresist may be used to separate some of the patches from some of the others or all of the patches from each other. Alternatively, non-bioreactive organic thinfilms, such as monolayers, with different wettability may be used to separate patches from one another.

Other examples of surface coatings and binding reagents are described in U.S. patent application Ser. Nos. 09/115,455, 09/353,215, and 09/353,555, and U.S. Pat. No. 6,454,924, which are herein incorporated by reference in their entirety for all purposes, and are assigned to the same assignee as the present application.

The terms and expressions which have been employed herein are used as terms of description and not of limitation, and there is no intention in the use of such terms and expressions of excluding equivalents of the features shown and described, or portions thereof, it being recognized that various modifications are possible within the scope of the invention claimed. Moreover, any one or more features of any embodiment of the invention may be combined with any one or more other features of any other embodiment of the invention, without departing from the scope of the invention. For example, any feature of the methods of analyzing a sample described above can be incorporated into any of the assemblies, chips, or systems without departing from the scope of the invention.

In addition, the patents and scientific references cited herein are incorporated by reference in their entirety.

### EXAMPLES

Introduction

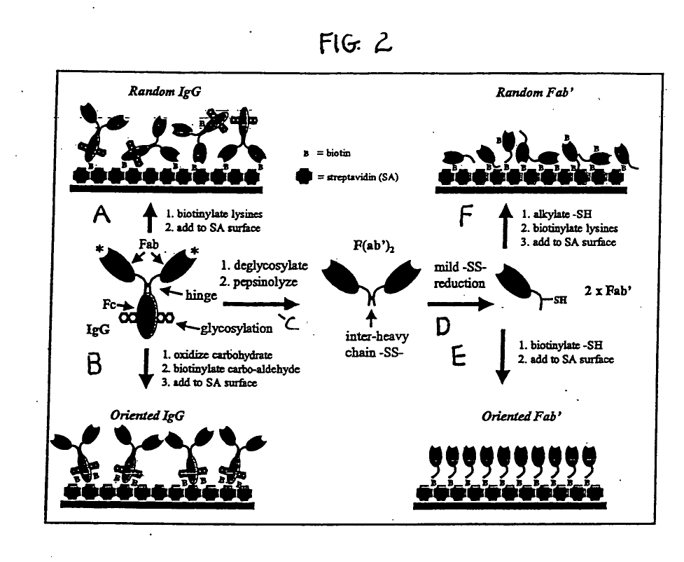

In these examples, four different antibody immobilization strategies are compared (FIG. 2). The four types are: randomly biotinylated IgG; oriented IgG (biotinylated on carbohydrate on Fc domain); oriented Fab′ fragments (biotinylated in hinge region) and randomly biotinylated Fab′ fragments. These four preparations of three different antibodies are compared to human cytokines in terms of surface density and binding activity. In addition, their performance in antigen-binding in a protein array format is examined.

Materials

MAB9647 is a mouse IgG₁ that was raised against the human IL-8; it was produced by Covance Inc. (Princeton, N.J.) from mouse ascites fluid using the hybrodoma cell line HB-9647 from ATCC (Manassas, Va.); it is protein G-purified. MAB208 is a mouse IgG₁ that was raised against human IL-8 by R&D Systems (Minneapolis, Minn.); it is protein G-purified from mouse ascites fluid, clone number 6217.111, catalog number MAB208. MAB602 is a mouse IgG_{2A} that was raised against human IL-2 by R&D Systems; it is protein G-purified from mouse ascites fluid, clone number 5355.111, catalog number MAB602. As a detection antibody for the IL-8 assays, an R-phycoerythrin (PE)-conjugated mouse anti-human IL-8 antibody was used (BD Pharmingen, cat. No. 20795A). For the IL-2 assays, two detection antibodies were used: a goat anti-human IL-2 antibody (R&D, cat. No. AF-202-NA) and an R-PE-conjugated donkey anti-goat IgG F(ab′)2 (Jackson Immuno Research, cat. No. 705-116-147). Unless noted, chemicals were purchased from Sigma-Aldrich (St. Louis, Mo.). Pepsin-agarose was purchased from Pierce (Rockford, Ill.), product number 20343. PNGase F was obtained from New England Biolabs (Beverly, Mass.), product number P0704. Aldehyde-reactive probe was from Molecular Probes (Eugene, Oreg.). Human interleukin-2 (IL-2) was purchased from Leinco Technologies (St. Louis, Mo.), product number 01 1R455, and was reconstituted in phosphate buffered saline (PBS; 11.9 mM sodium phosphate, 137 mM NaCI, 2.7 mM KCl, pH 7.4). A DNA sequence encoding the mature version of human Interleukin-8 (IL-8; AVLPRSAKELRCQCIKT′YSKPFHPKFIKELRVIES GPHCANTEIIVKLSDGRELCLDPKENWVQRVVEKFLKRAENS) SEQ ID 1 with a C-terminal Factor Xa cleavage site and Protein Kinase A site (GIEGRRRASV) SEQ ID 2 was created by gene assembly of oligonucleotides. This construct was inserted into the NdeI and XhoI sites of pET24a (Novagen, Madison, Wis.), resulting in the further addition of a His(6) tag at the extreme C-terminus (LEHH SEQ ID 3; where the LE codons comprise the XhoI site). This plasmid was then used to direct expression of IL-8 in BL21 DE3 cells (Novagen) grown in EZMix modified Terrific Broth (Sigma, St. Louis, Mo.; CAT No. T-9179) by the addition of 1 mM IPTG at an OD of 0.6 and growth for four hours in a BioFlo3000 fermentor (New Brunswick Scientific, Edison, N.J.) at 30° C. The cells were collected by centrifugation, resuspended in 5 ml buffer/g with 300 mM NaCl, 50 mM sodium phosphate, 5 mM beta-mercaptoethanol, 5 mM imidazole, pH 8.0 including one Complete Protease Inhibitor Cocktail tablet (Roche Applied Science; Cat No. 1697498) per 50 ml and lysed using a microfluidizer. The soluble fraction of IL-8 was purified by metal affinity chromatography using TALON Superflow beads (Clontech, Palo Alto, Calif.; Cat No. 8908-2) followed by gel filtration in PBS using a Superdex 75 prep grade column (Amersham Biosciences). The IL-8 containing fractions were concentrated to 0.4-0.5 mg/ml using a model 8400 stirred ultrafiltration cell (Millipore, Bedford, Mass.; CAT No 5124) with a 3K MWCO membrane and then dialyzed into PBS with 10% glycerol for storage. Identity and Correct secondary structure was confirmed by mass spectrometry, ELISA and circular dichroism (Spectrapolarimeter J-810, Jasco, Easton Md., www.jascoinc.com).

Preparation of Biotinylated Fab′ Fragments

The conserved N-linked glycosylation of IgG's were removed from the antibodies under the following reaction conditions: 1-4 mg/ml antibody in 50 mM sodium phosphate, pH 7.5, 10-20 U/μl PNGase F (from New England Biolabs, using their unit definition), 24 48 h at 37° C. After deglycosylation, antibodies were buffer-exchanged (using ultrafiltration or dialysis) into 20 mM sodium acetate, pH 4.5. Conditions for pepsinolysis were as follows: 30% (by volume) pepsin agarose (settled bed volume, beads washed in 20 mM NaOAc, pH 4.5), 0.5-2 mg/ml IgG, 20 mM NaOAc, 260 mM KCl, 0.1% Triton X-100, pH 4.5. Reactions were incubated at 37° C. with agitation for an amount of time that had previously been optimized (MAB9647, 12 h; MAB208, 4.5 h; MAB602, 3.5 h). After pepsin-treatment, the fragments were recovered from the pepsin agarose by washing the resin with 0.1M NaOAc, pH 4.5. The products of the pepsin-cleavage were then concentrated and exchanged into 0.1 M Na₂HPO₄, 5 mM EDTA, pH 6.0, and then treated with 20 mM 2-mercaptoethylamine (MEA) in the same buffer for 90 min at 37° C. The MEA was then removed by dialyzing for 6 hours at 4° C. against 0.1 M Na₂PO₄, 5 mM EDTA, using a 10 kD cutoff membrane, and then residual MEA was removed by running sample over a desalting column (PD-10, Amersham-Pharmacia, Piscataway, N.J.). Immediately after this step, the reduced F(ab)′ was treated with 20 mM N-ethylinaleimide or maleimide-activated biotin (Pierce product number 21901) for 2 h at room temperature, and the unincorprated NEM or biotin-maleimide was then removed by dialysis. The samples were concentrated and the Fab′ fragments were purified from other fragments by FPLC using a Superdex-75 gel filtration column (Amersham-Pharmacia). In the case of the NEM-treated Fab′ fragments, random biotinylation was as described below.

Samples of the FPLC-purified Fab′ fragments were diluted 1:1 with non-reducing protein loading buffer (62.5 mM TrisHCl, 25% glycerol, 2% SDS, 0.01% Bromphenol Blue) and loaded onto a 4-20% gradient SDS polyacrylamide gel (Product number 161-1123, Biorad, Hercules, Calif.). SDS-PAGE was performed according to Laemmli (1970). In each case the 50 kD band corresponding to the Fab′ was observed, and the only observable contaminants correspond to the sizes of the light and cleaved heavy chains. Because these contaminants co-migrated with the Fab′ in a high resolution gel filtration column, they presumably correspond to a non-covalent but otherwise structurally native complex between the light and cleaved heavy chains, in which the disulfide bond that normally links them has been reduced and alkylated. These fragments are likely to be functional since the disulfide bond between the heavy and light chain is not in the antigen-binding site. They correspond to about 20% of the purified protein, and some of these Fab′ fragments are >80% active (see below).

The biotinylation of all capture agents used in this study was verified by Western blot analysis using an HRP-conjugated streptavidin (SA) probe (data not shown). In addition, the extent of biotinylation was estimated by using a SA resin pull-down assay (data not shown). Biotinylation was 60% or greater in each of these reactions. No attempt was made to remove non biotinylated protein prior to surface immobilization.

Carbobiotinylation of IgG's

IgG (3-5 mg/ml) were dialyzed into coupling buffer (0.1 M NaOAc pH 5.5) and then incubated with 20 mM sodium meta periodate, NaIO₄ in the dark for 1 h at 0° C. to oxidize the vicinal diols in the carbohydrate to aldehydes. The reaction was then quenched by the addition of 30 mM glycerol for 10 minutes, filtered to remove insoluble salts, and then dialyzed against coupling buffer for 6 hours. The aldehyde reactive probe (ARP, N-(animooxyacetyl)-N′-(D biotinyl) hydrazine, trifluoroacetic acid salt, Molecular Probes, Eugene, Oreg.) is added at 1 mg/ml and incubated at room temperature for 2 h, after which the sample is extensively dialyzed against PBS.

Random Biotinylation of IgG and Fab′ Fragment Molecules

IgG and NEM-treated Fab′ fragments were modified with the amine reactive probe, EZ Link™ Sulfo-NHS-Biotin (Pierce). Reactions were performed with a 20-fold molar excess of biotinylation reagent over protein in 1×PBS at room temperature for 2 hours. The biotinylation reagent was then quenched by adding Tris, pH 7.4, to a final concentration of 10 mM. The samples were then dialyzed against a 1000-fold excess of PBS 5 times in order to remove free biotin probe. After dialysis, the biotinylated proteins were analyzed on a gel and tested for extent of biotinylation on UltraLink™ Plus Immobilized Streptavidin Gel (Pierce). Typically 60% -100% of the protein would be biotinylated (data not shown).

BIAcore Studies to Measure Surface Coverage and Activity

All surface plasmon resonance (SPR) assays were performed in a BIAcore 3000 using a biotinylated self-assembled monolayer formed on a gold-coated glass surface by immersion in an ethanolic solution of an unsymmetrical alkanedisulfide. The omega functionalities of the oligo(ethyleneglycol)-containing alkane disulfides are N-hydroxysuccinimide and methoxy groups. This monolayer was then reacted with tri(ethylene glycol) amino biotin to give a biotinylated surface (manuscript in preparation). We refer to this surface as biotinylated self-assembled monolayer (b-SAM). The biotin groups on the surface allow for the binding of streptavidin (SA). All assays were performed at 25° C. in PBS with 0.05% Tween-20. SA was loaded onto the surface at a flow rate of 20 μl/min at 0.1 mg/ml. Typically 320 μl was loaded to achieve a saturated surface of SA whereby a surface coverage of 3.7-4.0 pmol/cm² was obtained, as calculated according to Jung et al., Langmuir 14:5636-5648 (1998). After SA deposition, the various capture agents were loaded at 20-100 nM at a flow rate of 20 μl/min until saturation was observed. For analyte binding, flow rates of 80 μl/min were used unless otherwise noted. Various analyte concentrations over a broad range were assayed in order to determine the upper limit of analyte binding. For comparison, non-specific binding of the analytes to SA was tested independently at all analyte concentrations studied.

Array Assays

Array assays were performed on the biotinylated-Poly-L-Lysine (PLL)-Poly(ethyleneglycol) (PEG) system as previously described (Ruiz-Taylor et al., Proc. Natl. Acad. Sci. U.S.A. 98:852-857 (2001); Ruiz-Taylor et al., Langmuir 17: 7313-7322 (2001)) on a chip with six separate flow cells, each containing 250 addressable features. Thirty percent of the PEG side chains are derivatized with biotin. After SA deposition, the surfaces were washed and assembled into the flow cell chamber set up as described in Ruiz-Taylor et al., Proc. Natl. Acad. Sci. U.S.A. 98:852-857 (2001). The various capture agents tested were then loaded into different flow cells by applying typically 150 μl of a 100 nM stock of each capture agent over the appropriate flow cell at a flow rate of 0.05 ml/min. After extensively washing the flow cells with PBS, analyte was flowed over the cells at the indicated concentrations at a flow rate of approximately 20 μl/minute and allowed to incubate for an additional 30 minutes.

For IL-8 assays, the analyte was diluted into Superblock/TBS (Pierce) with 0.05% Tween-20. Unbound analyte was rinsed out of the flow cell by applying 0.5 ml of 1×PBS with 0.05% Tween-20 and 350 mM NaCl over the flow cells over a 5 minute period. This was followed by addition of 200 μl of PE-conjugated anti-IL-8 secondary antibody (10 nM) over each flow cell in Superblock with 0.05% Tween-20 and 250 nM mouse IgG Fc as a blocking agent. The secondary antibody was passed over the flow cell at a flow rate of 20 μl/minute and allowed to incubate for an additional 30 minutes. The flow cells were rinsed with 1×PBS with 0.05% Tween-20 and 350 mM NaCI over a 5 minute period. The microarray chips were then removed from the flow cell apparatus and quickly rinsed with 25 ml of 1×PBS followed by 10 ml of H₂O. The chips were then analyzed in a GSI Lumonics® 5000XL microarray scanner. Data was then quantified using the QuantArray software (GSI Lumonics).

For IL-2 assays, the conditions were similar to those for IL-8, except that the analyte was in 1×PBS with 3% non-fat milk and 0.05% Tween-20 during the incubation with the surfaces. In addition, the IL-2 assays were based on a tripartite antibody sandwich system which employed a goat anti IL-2 antibody (R&D Systems) followed by an anti goat PE-F(ab′)₂ (Jackson Immuno Laboratories). Secondary and tertiary antibodies were used at 10 nM concentration in 1×PBS with 3% non-fat milk, 0.05% Tween-20, and 250 nM mouse IgG Fc as a blocking agent. Incubations were performed as described for the IL-8 detection antibody with an extra wash step in between the two antibody incubations.

Results

The four immobilization strategies for the capture agents used in this study are shown in FIG. 2. The structure of the monoclonal IgG is shown on the middle portion of the diagram. It consists of two antigen binding (Fab) fragments connected by a hinge region to the Fc portion, which is usually glycosylated. The antigen binding face of the Fab's are marked by asterices. The IgG's can be randomly biotinylated on lysine residues by NHS-biotin (A). This results in heterogeneity of the position and number of biotin molecules on each antibody, and thus of the orientation on the streptavidin (SA)-derivatized sensor surface. To orient the IgG's, the conserved N-linked glycosylation site on the Fc portion can be oxidized with periodate, and subsequently biotinylated using the biotin-amino-oxy compound (N-aminooxyacetal)-N′-(D-biotinoyl)hydrazine (ARP) (B). This allows for homogeneous orientation of the IgG's on the surface, in which the point of attachment is distal from the antigen-binding site. Alternatively, the Fc region can be removed by proteolysis. To accomplish this, it is first deglycosyated and then treated with pepsin (C). The resulting F(ab′)₂ fragment is then reduced to monomeric Fab′ fragments with 2-mercaptoethylamine (D). The reduced cysteine thiols on the hinge region can then be modified with biotin maleimide, which allows for oriented attachment of the Fab′ fragments on the SA surface (E). Since the hinge region is on the opposite side of the Fab′ fragment from the antigen binding site, the latter are oriented away from the surface. To test the importance of the orientation, the Fab′ fragments can also be alkylated and then randomly biotinylated, thus giving randomly oriented Fab′ fragments on the SA surface (F).

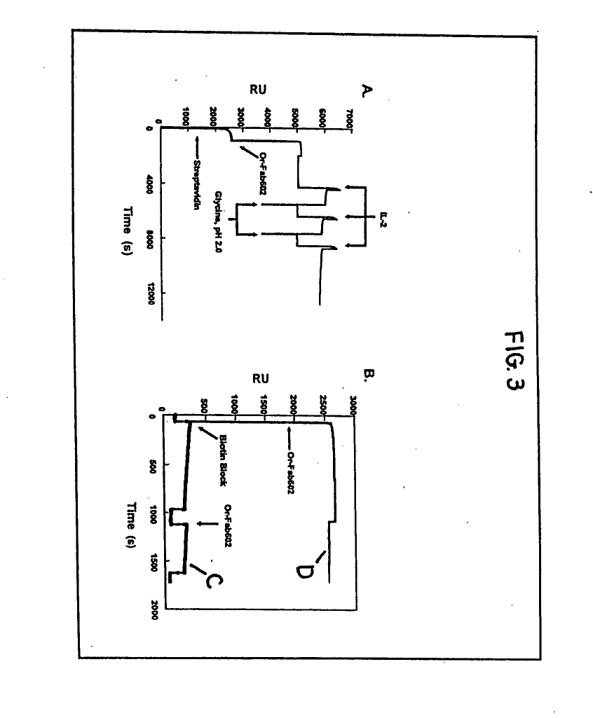

Biotin-dependent deposition of streptavidin and biotinylated-antibodies onto the alkane-thiol-biotin monolayer and the subsequent binding of analyte by the capture agent is shown in FIG. 3. Panel A shows a typical sensogram from a BIAcore experiment which demonstrates the sequential loading of streptavidin, oriented (Or) Fab′602, and IL2 onto b-SAM monolayer, as indicated by the arrows. Reactions were performed in 1×PBS with 0.05% Tween-20 at room temperature. For the binding of streptavidin (0.1 mg/ml stock) and Or-Fab′602 (50 nM stock) flow rates of 20 μl/min were employed. For IL2 binding steps, reactions were carried out at 40 μl/min. To titrate the active sites, IL2 was assayed at 600 nM so as to be at least >100-fold above the K_{d} (approximately 0.1 nM, data not shown). The antibody surface was regenerated with a 30 second injection of glycine pH 2.0 solution, as indicated by the arrows, to release any remaining analyte. As shown in Panel A, the surface could be regenerated to nearly 100% activity as assessed by the SRP measurements. (B) After deposition of the typical amount of streptavidin (not shown), 50 nM Or-Fab′602 was injected with (C) or without (D) a preinjection of 1 mM free biotin. Preinjection of free biotin blocked the loading of the antibodies. This was typically seen for all the other antibodies as well (data not shown).

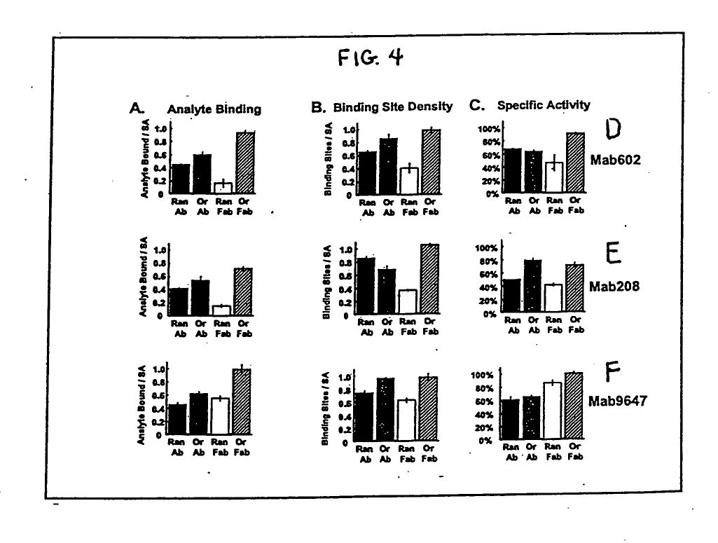

The effects of the various linkage strategies on binding site densities and binding activities are shown in FIG. 4. From BIAcore assays similar to those shown in FIG. 3, the surface densities of the various antibodies and their respective percentage activities were determined. For each experiment, the surface coverage of streptavidin (SA), antibody, and analyte were calculated separately from the appropriate SPR measurements (Jung et al., Langmuir 14:5636-5648 (1998)). The deposition of SA onto the b-SAM monolayer was very consistent for each experiment, typically ranging between 3.7-4.0 pmol/cm² (data not shown). To standardize the data from each experiment, ratios of capture agent/SA and analyte/(# of binding sites) were computed from the surface coverage values in order to compare the relative binding site densities and activities for the various types of linked capture agents. A valency of two for the full length antibodies and one for the Fab′ fragments was assumed in calculating the Binding Site/SA ratio in Panel B. This was done for all three capture agents: MAB602, MAB208, and MAB9647 as indicated on the right of each panel (represented by D, E, and F, respectively). In order to saturate the active sites, 100 nM target analyte concentrations were employed for MAB208 and MAB602 while a 1 μM analyte concentration was used for 9647. For each antibody, target anal binding was measured for the oriented glycosylated antibody (Or Ab), the randomly attached glycosylated antibody (Ran Ab, the oriented Fab (Or Fab), and the randomly attached Fab (Ran Fab)

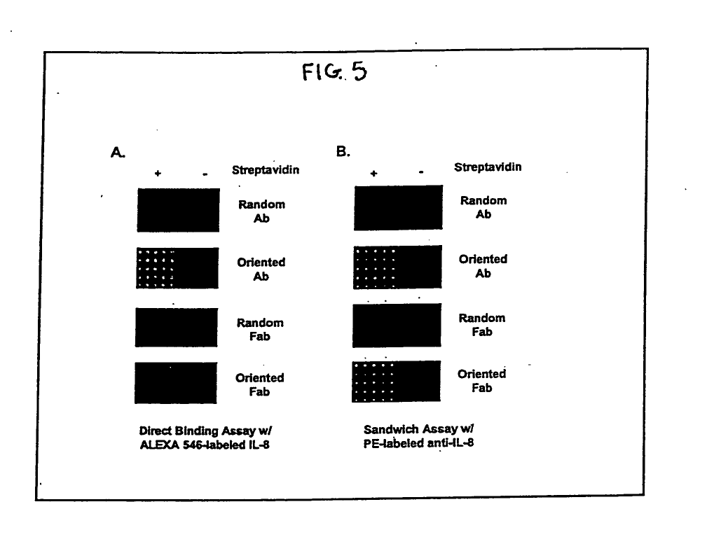

The effects of the various linkage strategies on MAB208 antibody performance on the PLL-PEG Biotin Microarray Matrix is shown in FIG. 5 (Panel A). Direct binding assay using 100 nM ALEXA 546-labelled hIL8. The various antibodies for each set of 5×5 pillars is labeled on the right. Adjacent to the 5×5 capture set is a control set of pillars on which were deposited PLL-PEG without biotin. The image was taken in a GSI Lumonics ScanArray® 5000XL with a Laser setting of 80 and a PMT setting of 80. (Panel B) Sandwich assay using 20 nM unlabelled hILB and 10 nM PE-conjugated anti-hIL8 detection antibody. As in panel A, the various antibody types are indicated at the right of each 5×5 set of microarray pillars. This image was also taken in the GSI Lumonics ScanArray® 5000XL using a laser setting of 70 and a PMT setting of 70. For both the A and B panels, all assays were conducted on a single chip and scanned simultaneously.

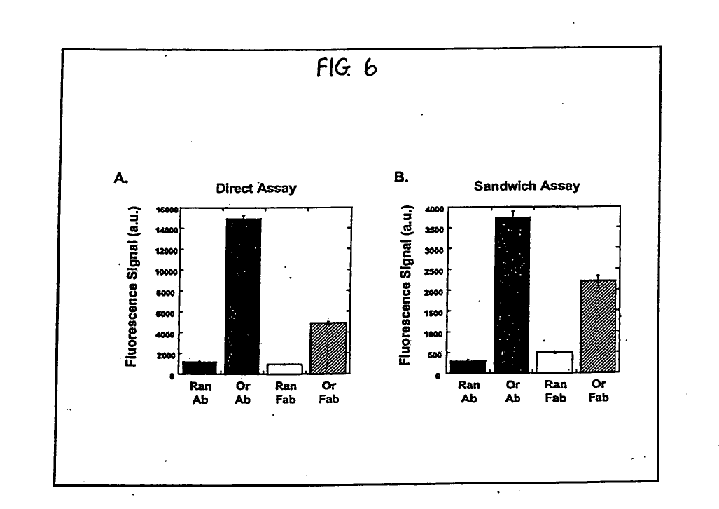

FIG. 6 shows the quantitation of the MAB208 direct binding and sandwich assays. Panel A show the plot of the average fluorescence intensities for the various MAB208 antibody forms from the direct binding assay in FIG. 5, panel A. Data represents the average intensity ±SEM (where N=25). Panel B show the plot of the average fluorescence intensities for the various MAB208 capture agent forms from the sandwich assay in FIG. 5, panel B. Data represents the average total intensity ±SEM (where N=25). All data were obtained through the Quantarray Software from GSI Lumonics.

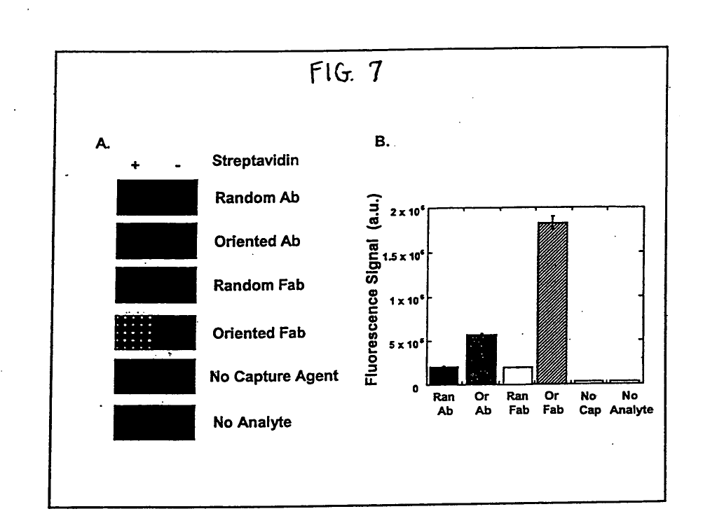

FIG. 7 shows a comparison of the four MAB208 antibody forms on the PLL PEG biotin microarray surface at 100 pm hIL8. Panel A shows the binding of 100 pM hIL8 to the various capture agent forms of MAB208 as monitored via a sandwich assay using 10 nM PE-conjugated anti-hIL8 (BD Pharmingen). Panel B shows the average intensities ±SEM, (where N=25) for the pillar sets in Panel A. Intensities were quantified using the Quantarray Software (GSI Lumonics). For both the A and B panels, all assays were conducted on a single chip and scanned simultaneously.

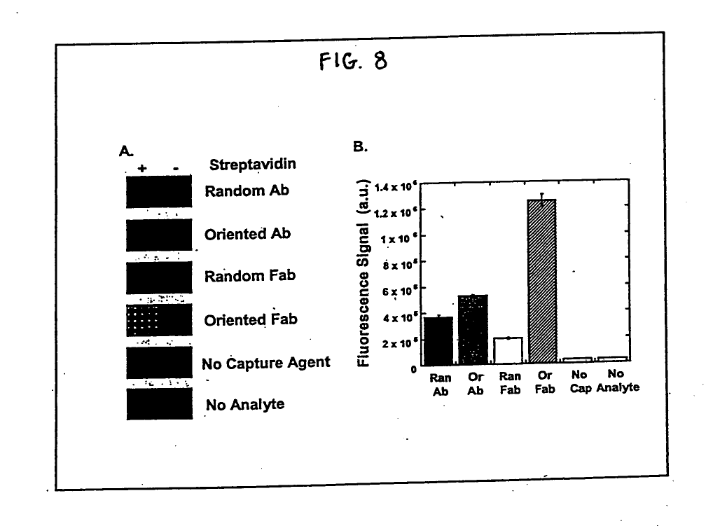

FIG. 8 shows a comparison of the four MAB602 antibody forms on the PLL PEG biotin microarray surface at 1 nM hIL2. Panel A shows the binding of 1 nM IL2 to the various capture agent forms of MAB602 as monitored via a tripartite sandwich assay using 10 nM goat anti-hIL2 and 10 nM PE-conjugated donkey anti-goat IgG. Panel B shows the average intensities ±SEM (where N=25) for the pillar sets in Panel A. Intensities were quantified using the QuantArray Software (GSI Lumonics).

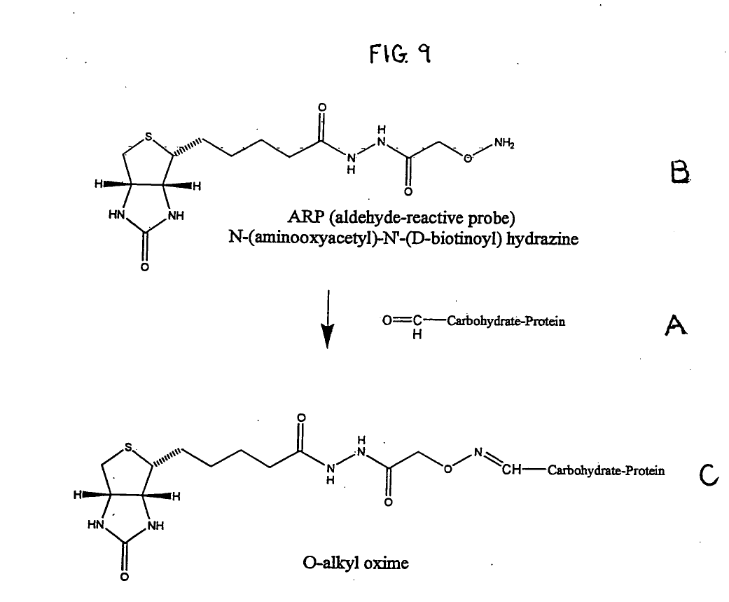

FIG. 9 depicts an exemplary embodiment of the invention where a glycoprotein (A) having at least one aldehyde or ketone group, and a biotin derivative containing an aminooxy-functionality (B), wherein the aminooxy functionality attacks the carbonyl carbon of the aldehyde or ketone group of the glycol-protein to form an O-alkyl oxime bond between biotin and the oxidized glycol-protein (C).

1. A method for conjugating a glycoprotein to a linking compound to form a glycoprotein linking compound comprising the steps of:
(a) oxidizing said glycoprotein to form an oxidized glycoprotein;
(b) contacting said oxidized glycoprotein with an aminooxy-functionalized linking compound;
(c) reacting said oxidized glycoprotein with said aminooxy-functionalized linking compound thereby forming said glycoprotein linking compound.

2. The method of claim 1, wherein said aminooxy-functionalized linking compound is attached to a surface prior to contacting said oxidized glycoprotein.

3. The method of claim 1, further comprising attaching said glycoprotein linking compound to a surface.

4. The method of claim 1 in which said glycoprotein is an antibody or an antibody-fragment.

5. The method of claim 1 in which said aminooxy-functionalized linking compound is a derivative of biotin.

6. The method of claim 5 in which said biotin derivative is N-(aminooxyacetyl)-N′-(Dbiotinoyl)hydrazine.

7. The method of claim 1 wherein said glycoprotein is a non glycosylated protein glycosylated to form said glycoprotein.

8. The method of claim 2 wherein said aminooxy-functionalized linking compound is attached to said surface through a monolayer.

9. The method of claim 3 wherein said aminooxy-functionalized linking compound portion is attached to said surface through a monolayer.

10. A method of forming an immobilized glycoprotein comprising the steps of:
(a) contacting a glycosyl moiety covalently bound to said glycoprotein with an oxidizing agent to form an oxidized glycosyl moiety;
(b) contacting said oxidized glycosyl moiety with an aminooxy-functionalized linking compound to form a glycoprotein linking compound;
(c) contacting said glycoprotein linking compound with an organic thinfilm to form an immobilized glycoprotein.

11. The method of claim 10, wherein said oxidizing agent is sodium meta peroidate.

12. The method of claim 10, wherein said functionalized linking compound comprises a biotin moiety and said organic thinfilm comprises a streptavidin moiety.

13. The method of claim 10, wherein said glycoprotein is an antibody or an antibody fragment.

14. The method of claim 10, wherein said organic thinfilm is a monolayer.

15. A glycoprotein immobilized on an organic thinfilm wherein the glycoprotein comprises a glycosyl moiety covalently bound to a linking compound through an oxime bond, wherein said linking compound is bound to said organic thinfilm.

16. An array of glycoproteins comprising a plurality of glycoproteins arranged in discrete, known regions on portions of an organic thinfilm, wherein each of said plurality of glycoproteins comprise a glycosyl moiety covalently bound to a linking compound through an oxime bond, wherein said linking compound is bound to said organic thinfilm.
